(19) United States
(12) Patent Application Publication
(10) Pub. No.: US 2018/0165004 A1
(43) Pub. Date: Jun. 14, 2018

(54) SYSTEM AND METHOD FOR INTERACTIVE 3D SURGICAL PLANNING AND MODELLING OF SURGICAL IMPLANTS

(71) Applicant: CONCEPTUALIZ INC., Little Britain (CA)

(72) Inventors: Richard HURLEY, Toronto (CA); Rinat ABDRASHITOV, Toronto (CA); Karan SINGH, Toronto (CA); Ravin BALAKRISHNAN, Little Britain (CA); James MCCRAE, Brampton (CA)

(21) Appl. No.: 15/895,324

(22) Filed: Feb. 13, 2018

Related U.S. Application Data
(63) Continuation of application No. 14/703,044, filed on May 4, 2015.
(60) Provisional application No. 61/989,232, filed on May 6, 2014.
(60) Provisional application No. 62/046,217, filed on Sep. 5, 2014.

(51) Int. Cl. G06F 3/0488; G06F 3/0484; G06F 3/0481

(52) U.S. Cl. CPC G06F 3/04883; G06F 3/04842; G06F 3/04815; A61B 2034/256; G06F 19/3437

(57) ABSTRACT

A method and system for interactive 3D surgical planning are provided. The method and system provide 3D visualisation and manipulation of at least one anatomical feature in response to intuitive user inputs, including gesture inputs. In aspects, fracture segmentation and reduction, screw placement and fitting, and plate placement and contouring in a virtual 3D environment are provided.

### TECHNICAL FIELD

The following relates to surgical planning, and more specifically to a system and method for interactive 3D surgical planning. The following further relates to interactive 3D modelling of surgical implants.

### BACKGROUND

Preoperative planning is indispensible to modern surgery. It allows surgeons to optimise surgical outcomes and prevent complications during procedures. Preoperative planning also assists surgeons to determine which tools will be required to perform procedures.

The value of preoperative planning has long been recognised, particularly in the field of orthopaedic surgery. In recent years, however, increased technical complexity and cost pressures to reduce operating room time have led to greater emphasis on preoperative planning.

One of the purposes of preoperative planning is to predict implant type and size. It is important that implants fit accurately and in the correct orientation. Frequently, a surgical team will prepare numerous implants of varying sizes to ensure that at least one will be appropriately sized for a surgical operation. The more accurately the team can predict the required implant configuration, the fewer implants required to be on hand during the operation; this reduces the demand for sterilisation of redundant tools and implants. More accurate predictions may also reduce operating time, thereby decreasing the risk of infection and patient blood loss.

A thorough preoperative plan includes a careful drawing of the desired result of a surgical operation.

Standard preoperative planning is typically performed by hand-tracing physical radiographic images or using digital 2D systems that allow manipulation of radiographic images and application of implant templates. More recently, 3D computed tomography (CT) reconstruction has been developed and has shown to be a useful adjunct in the surgical planning of complex fractures.

Several preoperative planning software solutions exist. The majority of such solutions are used by surgeons prior to surgery at a location remote from the surgery.

### SUMMARY

In an aspect, a system for segmentation and reduction of a three-dimensional model of an anatomical feature is provided, the system comprising: a display unit configured to display a two-dimensional rendering of the three-dimensional model to a user; an input unit configured to receive a user input gesture comprising a two-dimensional closed stroke on the display unit; and a manipulation engine configured to: select a subset of the three-dimensional model falling within the two-dimensional closed stroke; receive a further user input gesture from the input unit; and manipulate in accordance with the further user input gesture the subset relative to the surrounding three-dimensional model from an initial placement to a final placement.

In an aspect, a method for segmentation and reduction of a three-dimensional model of an anatomical feature is provided, the method comprising: displaying, on a display unit, a two-dimensional rendering of the three-dimensional model to a user; receiving a user input gesture comprising a two-dimensional closed stroke on the display unit; selecting a subset of the three-dimensional model falling within the two-dimensional closed stroke; receiving a further user input gesture; and manipulating in accordance with the further user input gesture the subset relative to the surrounding three-dimensional model from an initial placement to a final placement.

In an aspect, a system for generating a three-dimensional model of a surgical implant for an anatomical feature is provided, the system comprising: a display unit configured to display a two-dimensional rendering of a three-dimensional model of the anatomical feature; an input unit configured to receive from a user at least one user input selecting a region on the three-dimensional model of the anatomical feature to place the three-dimensional model of the surgical implant; and a manipulation engine configured to generate the contour and placement for the three-dimensional model of the surgical implant in the selected region.

In an aspect, a method for generating a three-dimensional model of a surgical implant for an anatomical feature is provided, the method comprising: displaying, on a display unit, a two-dimensional rendering of the three-dimensional model of the anatomical feature; receiving from a user at least one user input selecting a region on the three-dimensional model of the anatomical feature to place the three-dimensional model of a surgical implant; and generating the contour and placement for the three-dimensional model of the surgical implant in the selected region.

In an aspect, a system for generating a two-dimensional rendering of a three-dimensional model of an anatomical feature from a plurality of datasets in response to a user input action from a user is provided, the system comprising: a display unit configured to display a plurality of parameters, the parameters corresponding to Hounsfield values; an input unit configured to receive a user input action from the user selecting at least one parameter corresponding to the Hounsfield value of the anatomical feature; and a modeling engine configured to retrieve a subset of imaging data corresponding to the at least one parameter and to generate a three-dimensional model of the anatomical feature therefrom, and further to generate a two-dimensional rendering of the three-dimensional model for display on the display unit.

In an aspect, a method for generating a two-dimensional rendering of a three-dimensional model of an anatomical feature from a plurality of datasets in response to a user input action from a user is provided, the system comprising: displaying a plurality of parameters, the parameters corresponding to Hounsfield values; receiving a user input action from the user selecting at least one parameter corresponding to the Hounsfield value of the anatomical feature; and retrieving a subset of imaging data corresponding to the at least one parameter and generating a three-dimensional model of the anatomical feature therefrom, and further generating a two-dimensional rendering of the three-dimensional model for display on the display unit.

In an aspect, a system for modeling screw trajectory on a three-dimensional model of an anatomical feature is provided, the system comprising: a display unit configured to display a two-dimensional rendering of the three-dimensional model to a user; an input unit configured to: receive a user input gesture from the user to modify the two dimensional rendering displayed by the display unit; and receive a user input action from the user indicating a desired screw location; and a manipulation engine configured to augment the three-dimensional model by applying a virtual screw to the three-dimensional model having a screw trajectory extending from the screw location to an end location perpendicularly into the three-dimensional model from the plane and at the location of the user input action.

In an aspect, a method for modeling screw trajectory on a three-dimensional model of an anatomical feature is provided, the method comprising: displaying a two-dimensional rendering of the three-dimensional model to a user; receiving a user input gesture from the user to modify the two dimensional rendering; receive a user input action from the user indicating a desired screw location; and augment the three-dimensional model by applying a virtual screw to the three-dimensional model having a screw trajectory extending from the screw location to an end location perpendicularly into the three-dimensional model from the plane and at the location of the user input action.

### BRIEF DESCRIPTION OF THE DRAWINGS

Features will become more apparent in the following detailed description in which reference is made to the appended drawings wherein:

FIG. 1 illustrates an embodiment of a system for interactive surgical planning;

FIGS. 2A to 2D illustrate a user interface for selecting, segmenting and manipulating a 3D model of an anatomical feature;

FIGS. 3A to 3D illustrate another user interface for selecting, segmenting and manipulating a 3D model of an anatomical feature;

FIGS. 4A to 4C illustrate a user interface for planning screw holes in a 3D model of an anatomical feature;

FIG. 5 illustrates a user interface for rearranging screw holes in the 3D model of the anatomical feature;

FIG. 6 illustrates embodiments of surgical plates;

FIG. 7 further illustrates embodiments of surgical plates and their segmented equivalents;

FIG. 8 illustrates a segment of an embodiment of a surgical plate;

FIG. 9 illustrates a 3D approximation of the segment of FIG. 8;

FIG. 10A illustrates a 3D approximation of an embodiment of a surgical plate composed of multiple segments;

FIG. 10B illustrates a 3D approximation of an embodiment of a surgical plate composed of multiple segments and comprising a drill guide;

FIGS. 11A to 11B illustrate a method for applying a discrete curve to the surface of a 3D model of an anatomical feature;

FIG. 12 further illustrates a method for applying a discrete curve to the surface of a 3D model of an anatomical feature;

FIGS. 13A to 13C illustrate a method for locating segment links on the discrete curve;

FIGS. 14A to 14C illustrate a method for arranging segment links along the discrete curve;

FIG. 15 illustrates a method for displaying and receiving angular coordinates;

FIG. 16 illustrates a user interface of a system for interactive surgical planning;

FIG. 17 illustrates a method for generating a 3D model of an anatomical feature;

FIG. 18 illustrates a method for manipulating the 3D model of an anatomical feature generated in FIG. 17;

FIG. 19 illustrates a method for planning screw and hole placement on the 3D model of an anatomical feature generated in FIG. 17; and

FIG. 20 illustrates a method for planning surgical plate placement on the 3D model of an anatomical feature generated in FIG. 17.

### DETAILED DESCRIPTION

Embodiments will now be described with reference to the figures. It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practised without these specific details. In other instances, well-known methods, procedures and components have not been described in detail so as not to obscure the embodiments described herein. Also, the description is not to be considered as limiting the scope of the embodiments described herein.

It will also be appreciated that any engine, unit, module, component, server, computer, terminal or device exemplified herein that executes instructions may include or otherwise have access to computer readable media, such as, for example, storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as, for example, computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Any application or module herein described may be implemented using computer readable/executable instructions that may be stored or otherwise held by such computer readable media. Such engine, unit, module, component, server, computer, terminal or device further comprises at least one processor for executing the foregoing instructions.

In embodiments, an intuitive system for interactive 3D surgical planning is provided. The system comprises: an input unit for receiving user input gestures; a manipulation engine for processing the user input gestures received in the input unit to manipulate a 3D model of at least one anatomical feature; and a display for displaying the 3D model manipulated in the manipulation engine.

In embodiments, the system provides an intuitive and interactive interface for surgical planning in three dimensions. The system further permits interaction with a 3D model of at least one anatomical feature to create a preoperative plan for patients. In embodiments, the system allows for surgical planning on a virtual model in real time using simple and intuitive gestures. Surgical planning may include: fracture segmentation and reduction; screw and plate placement for treating fractures; and planning of positioning of implants for treating a patient.

In further embodiments, a method for interactive 3D surgical planning is provided. The method comprises: in an input unit, receiving from a user at least one input gesture; in a manipulation engine, processing the at least one user input gesture received in the input unit to manipulate a 3D model of at least one anatomical feature; and in a display unit, displaying the 3D model manipulated in the manipulation engine.

In embodiments, the method provides intuitive and interactive surgical planning in three dimensions. The method further permits interaction with anatomical features to create a unique preoperative plan for patients. In embodiments, the method allows for surgical planning on a virtual model in real time using simple and intuitive input gestures.

In aspects, an intuitive method for interactive 3D surgical planning is provided.

In further embodiments, the system provides an intuitive and interactive interface for generating digital 3D models of surgical implants, including, for example, surgical joints, plates, screws and drill guides. The system may export the digital 3D models for rapid prototyping in a 3D printing machine or manufacture. The system may also export 3D models of anatomic structures, such as, for example, bone fractures, for rapid prototyping.

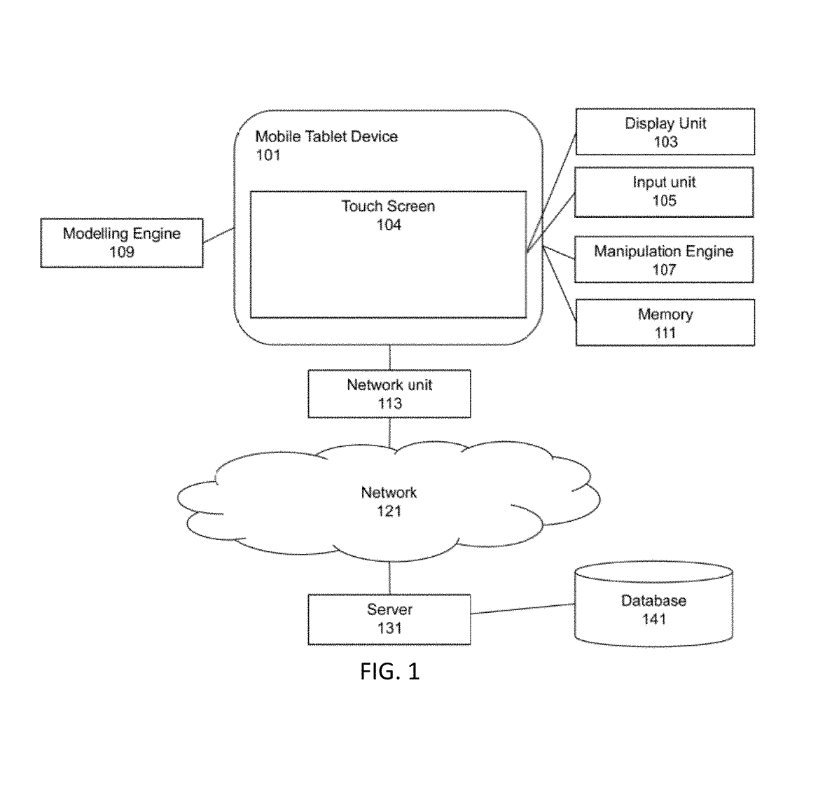

Referring now to FIG. 1, an exemplary embodiment of a system for interactive and 3D surgical planning is depicted. In the depicted embodiment, the system is provided on a mobile tablet device. For various reasons that will become apparent in the following description, the utilization of a mobile tablet device enables several advantages to the present system for a surgeon conducting a surgery. For example, a surgeon operating in a sterile environment may use a mobile tablet device encased in a sterile encasing, such as a sterile plastic bag, to view and interact with the generated preoperative plan. Notwithstanding the foregoing, the following is not limited to use on a mobile tablet device.

The mobile tablet device depicted in FIG. 1 has a touch screen 104. Where the mobile tablet device comprises a touch screen 104, it will be appreciated that the display unit 103 and the input unit 105 are integrally formed as a touch screen 104. In alternate embodiments, however, the display unit and the input unit are discrete. In still further embodiments, the display unit and some elements of the user input unit are integral, but other input unit elements are remote from the display unit. Together, the user input unit 105 and the display unit 105 present an interactive user interface to the user. The user input unit 105 and display unit 103 will be hereinafter described in greater detail. The use of a touch screen instead of a conventional input device in the embodiments described herein may facilitate increased interactivity, increased accessibility for 3-D surgical planning, intuitive direct manipulation of elements, simple control gestures, a reduced learning curve, and a flexible and dynamic display.

In embodiments, the mobile tablet device may comprise a network unit 113 providing, for example, Wi-Fi, cellular, 3G, 4G, Bluetooth and/or LTE functionality, enabling network access to a network 121, such as, for example, a secure hospital network. A server 131 may be connected to the network 121 as a central repository. The server may be linked to a database 141 for storing digital images of anatomical features. In embodiments, database 141 is a hospital Picture Archiving and Communication System (PACS) archive which stores 2D computerised tomography (CT) in Digital Imaging and Communications in Medicine (DICOM) format. The PACS stores a plurality of CT datasets for one or more patients. The mobile tablet device 101 is registered as an Application Entity on the network 121. Using DICOM Message Service Elements (DIMSE) protocol, the mobile tablet device 101 communicates with the PACS archive over the network 121.

The user of the system can view on the display unit 103 the available CT datasets available in the PACS archive, and select the desired CT dataset for a specific operation. The selected CT dataset is downloaded from the database 141 over the network 121 and stored in the memory 111. In embodiments, the memory 111 comprises a cache where the CT datasets are temporarily stored until they are processed by the modelling engine 109 as hereinafter described.

In embodiments, each CT dataset contains a plurality of 2D images. Each image, in turn, comprises a plurality of pixels defining a 2D model of an anatomical feature. Each pixel has a greyscale value. The pixels of a given anatomical feature share a range of greyscale values corresponding to a range of Hounsfield values. The CT datasets further contain at least the following data: the 2D spacing between pixels on each image, the position and orientation of the image relative to the other images, spacing between images, and patient identifiers, including a unique hospital identifier.

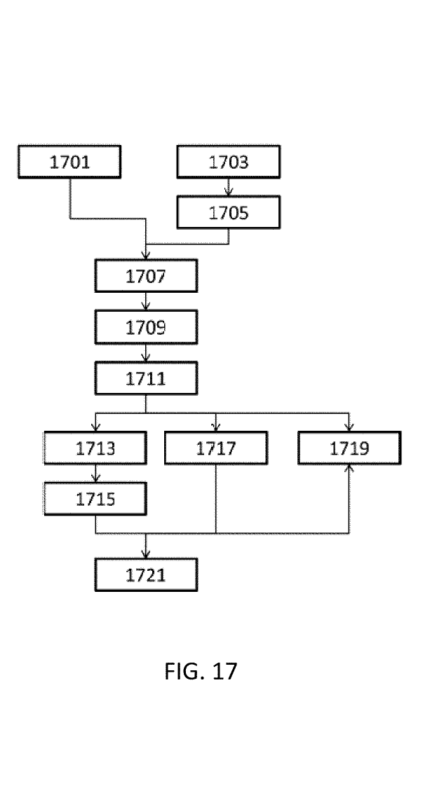

A method of generating a 3D model is illustrated in FIG. 17. At block 1701, the modelling engine 109 directs the display unit 103 to prompt the user to select a Hounsfield value corresponding to a desired anatomical feature. In embodiments, at block 1703 the modelling engine 109 retrieves from memory a pre-configured list of Hounsfield values and/or ranges of Hounsfield values and at block 1705 directs the display unit 103 to display the pre-configured list. The list preferably comprises Hounsfield values and/or ranges of Hounsfield values corresponding to particular categories of anatomical features such as, for example, bone, vessels and tissue, which can be configured based on known Hounsfield data for such features. It will be appreciated that the pre-configured list may improve the user experience, such as, for example, by presenting a preconfigured range of Hounsfield values that has been shown to accurately correspond to a given type of anatomical feature. At block 1707, the modelling engine 109 receives from the input unit 105 the Hounsfield value or range of Hounsfield values selected by the user.

At block 1709, the modelling engine 109 then retrieves from the dataset located in the memory 111 the data for the pixels corresponding to the selected Hounsfield value; all pixels having a greyscale value falling within the corresponding range of Hounsfield values are selected. As previously described, the dataset comprises: the 2D spacing between pixels on each image, the position and orientation of the image relative to the other images, and spacing between images. It will be appreciated that the dataset therefore contains sufficient information to determine in three dimensions a location for each pixel relative to all other pixels. The modelling engine 109 receives from the memory 111 the 2D coordinates of each pixel. At block 1711, the modelling engine 109 calculates the spacing in the third dimension between the pixels and thereby provides a coordinate in the third dimension to each pixel. At block 1719, the modelling engine 109 stores the 3D coordinates and greyscale colour for each pixel in the memory 111.

In embodiments, at block 1713 the modelling engine 109 generates a 3D model comprising all the selected points arranged according to their respective 3D coordinates. For example, the 3D model may be generated using the raw data as a point cloud; however, in embodiments, as shown at block 1715, the modelling engine 109 applies any one or more volume rendering techniques, such as, for example, Maximum Intensity Projection (MIP), to the raw data 3D model. At block 1721, the modelling engine 109 directs the display unit to display the 3D model.

It will be further appreciated, however, that the 3D model may be generated as a polygon mesh, as shown at block 1717. In still further embodiments, point cloud and polygon mesh models are both generated and stored. The modelling engine 109 transforms the 2D CT dataset into a polygon mesh by applying a transform or algorithm, such as, for example, the Marching Cubes algorithm, as described in William E. Lorenson and Harvey E. Cline, “Marching Cubes: A High Resolution 3D Surface Construction Algorithm” (1987) 21:4 Computer Graphics 163, incorporated herein by reference.

It will be appreciated that a polygon mesh comprises a collection of vertices, edges and faces. The faces consist of triangles. Every vertex is assigned a normal vector. It will be further appreciated that the polygon mesh provides for 3D visualisation of 2D CT scans, while providing an approximation of the curvature of the surface of the anatomical feature.

In embodiments, the modelling engine 109 generates a point cloud model, at block 1713, and a polygon mesh model, at block 1717, of the selected anatomical feature; these models are stored in the memory 111 of the mobile tablet device 101 for immediate or eventual display. Preferably, the models are retained in the memory until the user chooses to delete them so that the 3D modelling process does not need to be repeated. The 3D models having been generated, the CT datasets and identifying indicia are preferably wiped from memory 111 to preserve patient privacy. Preferably, the unique hospital identifier is retained so that the 3D models can be associated with the patient whose anatomical feature the 3D models represent.

In embodiments, 3D modelling is generated external to the mobile tablet device 101, by another application. The 3D models thus generated are provided to the mobile tablet device 101 over the network 121. In such embodiments, it will be appreciated that the CT datasets do not need to be provided to the mobile tablet device 101, but rather to the external engine performing the 3D modelling.

In embodiments, the 3D model is displayed on the display unit 103, preferably selectively either as a point cloud or polygon mesh. A user may then manipulate the 3D models as hereinafter described in greater detail.

In embodiments having a touch screen 104, as shown in FIG. 1, a user can manipulate the 3D depiction by using manual input gestures. For example, the user may: touch and hold (pan) with one finger the 3D depiction in order to rotate the depiction about any axis (i.e., free form rotation) or, selectively, about any one of the sagittal, coronal and transverse axes; zoom in and out by pinching two fingers apart and together on the touch screen 104, respectively and vice versa; draw a line by panning a single finger across the touch screen. It will be appreciated that providing for user input gestures to manipulate a 3D model of anatomical features enables intuitive and interactive visualisation. It will be further appreciated that selective manipulation of elements of an anatomical feature provides intuitive and interactive segmentation and reduction of the elements as is required in some surgeries, such as, for example, orthopaedic surgery.

In further embodiments, a settings menu is displayed on the touch screen 104. The settings menu may selectively provide the following functionality which, in some instances, are described in more detail herein manual input gesture control as previously described; selection of models available to be viewed, such as with a user interface button (“UI”) labeled “series”; surface and transparent (x-ray emulation) modes, such as with a UI button labeled “model”, wherein the x-ray emulation may provide simulated x-ray imaging based on the current viewpoint of the 3D model; an option to reduce model resolution and improve interactive speed, such as with a UI button labeled “downsample”, wherein, as described below, when a user performs any transformation the system draws points instead of the mesh so that the system may be more responsive, but once a user discontinues the associated user input, such as by releasing their fingers, the mesh is immediately drawn again; an option to enable a user to perform lasso selection, such as with a UI button labeled “selection or segmentation”, allowing a user to reduce, delete or crop a selection; an option to select the type of implant to be used (for example, a. screw, plate, hip, knee, etc.) such as with a UI button labeled “implants”; an option to select a measurement tool (for example, length, angle, diameter, etc.) such as with a UI button labeled “measurement”; an option to display the angle of the screen in relation to orthogonal planes, such as with a UI button labeled “screen view angle”; an option to select between anterior, posterior, left and right lateral, superior (cephalad), inferior (caudad) positions, such as with a UI button labeled “pre-set anatomical views”; an option to allow a user to easily take a screen shot that will be saved to photo library on device, such as with a UI button labeled “screenshot”; an option to allow a user to evaluate implant models in 1:1 ratio real life size on screen with present views as described above, and to export as a StereoLithography (“STL”) file to email or share through a digital file sharing medium (for example, Dropbox™, etc,) such as with a UI button labeled “export view”; an option to allow a user to check implant/bone interface fit thereby validating implant size and position and correlate with 2D orthogonal plane views, such as with a UI button labeled “interface fit or cut-away view”; an option to allow a user to unlock or lock screen rotation, such as with a UI button labeled “accelerometer”. Further, radial menus can be implemented to facilitate for touch inputs.

The foregoing functionality may enhance the user experience by, for example, allowing the user to more quickly or accurately recall preset views or to visualise environmental features that may impact the surgical procedure being planned.

Further, to provide the foregoing functionality, rendering of 3D models can be decoupled from touch inputs, which may increase responsiveness. Specifically, when the user's input causes a transformation, the systems can be configured to draw points instead of an associated mesh and to only draw the mesh when the touch input is discontinued.

The described method of generating a 3D model may provide models having a relatively high resolution. The mesh used may be the raw output of the marching cubes algorithm, without downsampling. For example, output of such methods may provide a pelvic model having 2.5 million polygons and a head model having 3.9 million polygons. Further, it will be appreciated that a 3rd party rendering library may not be utilized.

In order to effect preoperative planning to, for example, treat bone fractures, a user may need to segment and select bones and fracture fragments. Once the bones and fracture fragments are segmented, the user can manually reduce them into anatomical position, as hereinafter described in greater detail. Where possible, the user can use as a template an unaffected area matching the treatment area to determine whether the user has properly reduced the fracture fragments.

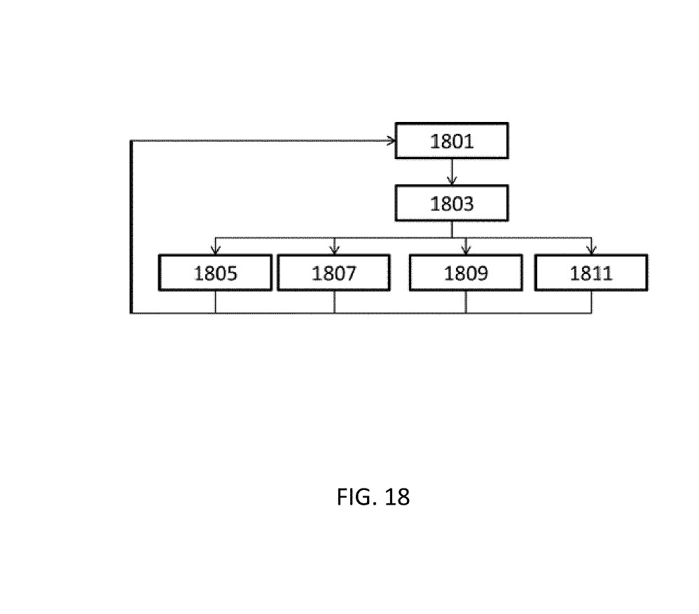

A method of segmenting the elements in a 3D model of an anatomical feature is shown in FIG. 18.

The user may manipulate the model to select an optimal view for segmenting the elements, as previously described. At block 1803 the user input 105 receives from the user a gesture input as previously described to manipulate the display of the 3D model. At block 1805 the manipulation engine 107 manipulates the display, at block 1801, of the 3D model. Once the user is satisfied with the display of the 3D model, the user draws a 2D closed stroke on the touch screen display unit 103 around an element to segment. In embodiments, a user may wish to segment an element such as, for example, a bone fracture.

As shown in FIG. 18, at block 1803 the input unit 105 receives the user input gesture and the manipulation engine 107 performs a procedure or procedures, described below, at block 1807 to effect cutting and segmentation for each of the point cloud and polygon mesh models. Preferably, when the user draws the 2D closed stroke to segment a bone fracture, the modelling engine 109 performs both procedures without requiring the user to re-segment the bone fracture.

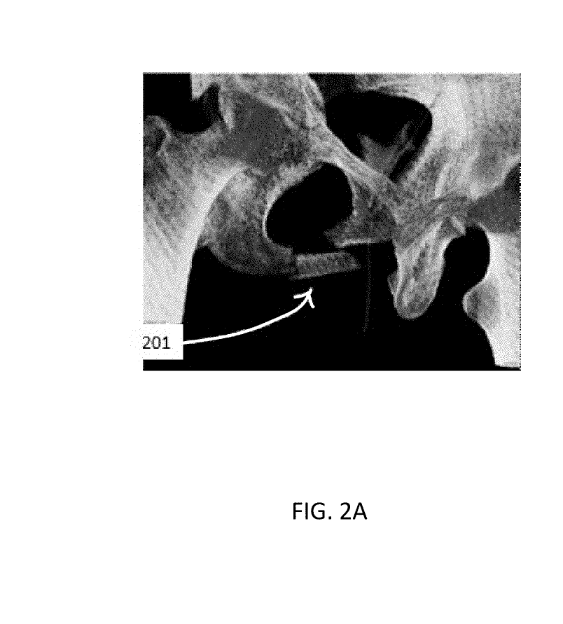

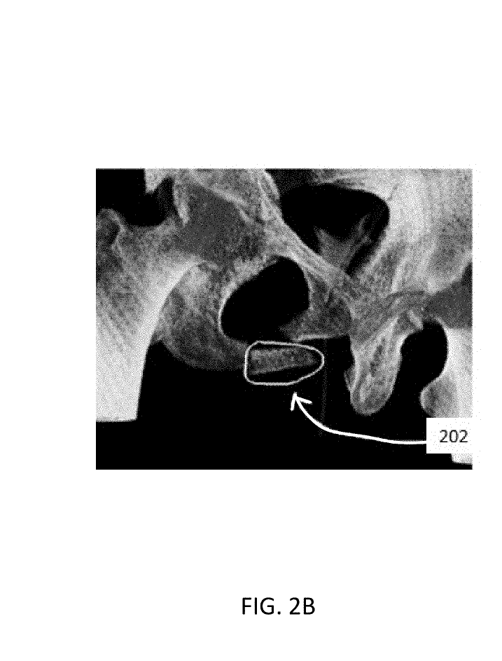

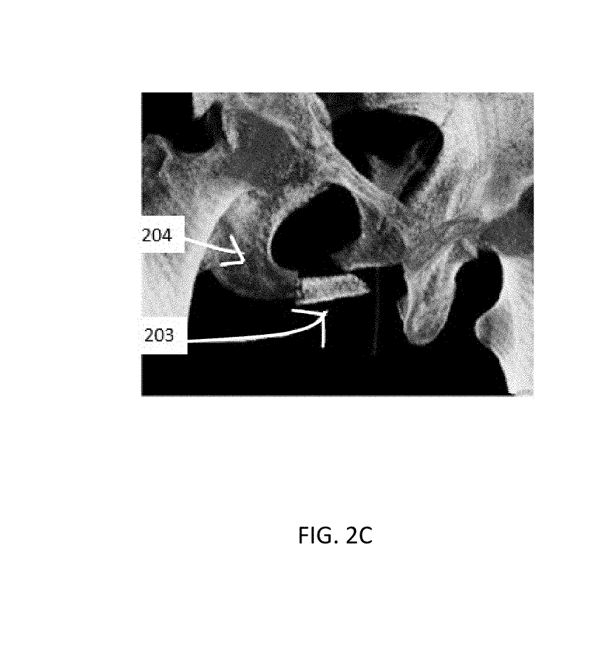

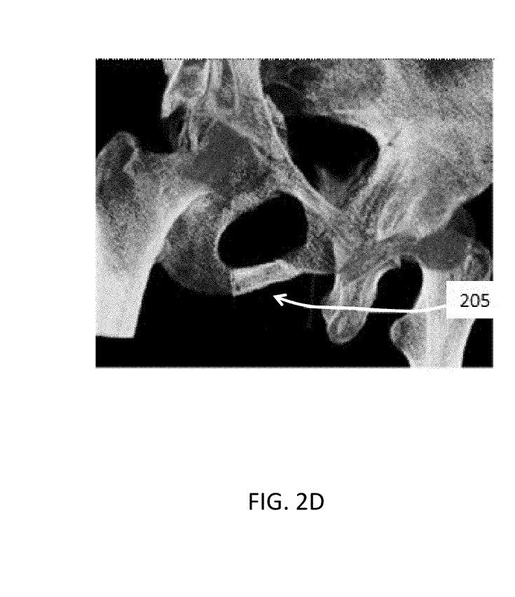

As shown in FIGS. 2A to 2D, a fractured anatomical feature is represented by a point cloud 3D depiction. The user first draws a 2D closed stroke 202 having 2D screen coordinates around fracture segment 201. Every 3D point having corresponding 2D screen coordinates falling within the 2D screen coordinates of closed stroke 202 will now be identified by the manipulation engine 107 as belonging to the selected fracture segment 203, at block 1807. The selected fracture segment 203, then, may be moved independently from, and relative to, the surrounding anatomical feature 204. Using the two finger panning input gesture to translate and the one finger panning input gesture to rotate, the user may manipulate the selected fracture segment 203 to a desired location 205 as depicted in FIG. 2D.

As shown in FIG. 18, at block 1803 input unit 105 receives the user input gesture and at block 1809, the manipulation engine 107 moves the segment in response to the user input gesture. The motion and final placement of the segment are displayed on the display unit 103 as shown at block 1801.

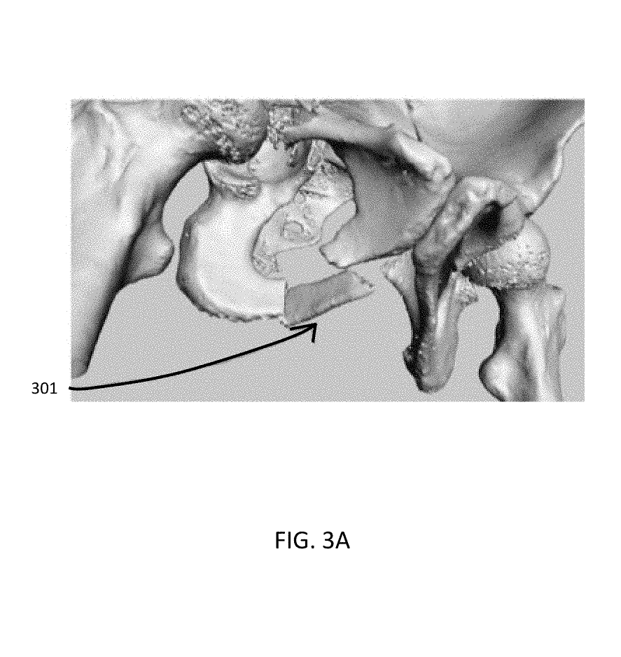

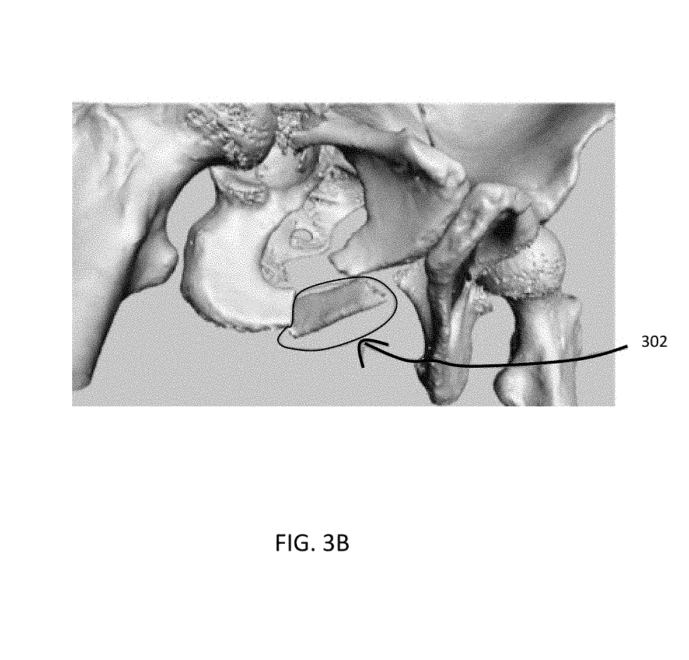

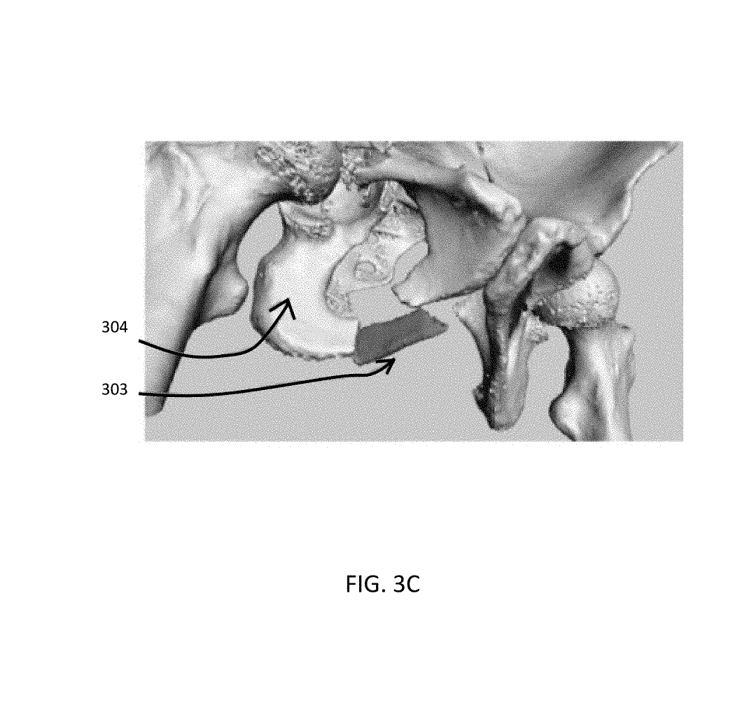

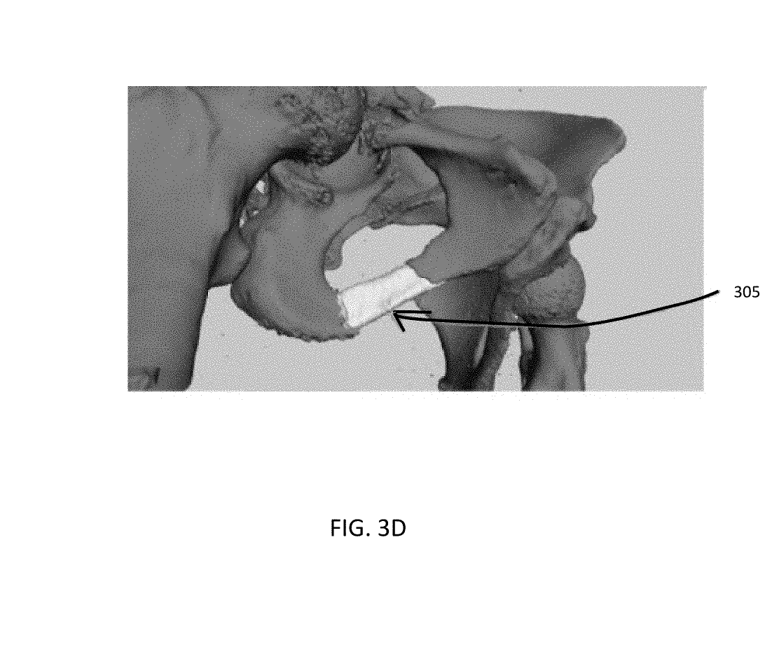

As shown in FIGS. 3A to 3B, a fractured anatomical feature is represented by a polygon mesh 3D depiction. The user draws a 2D closed stroke 302 around the fracture segment 301. The 2D closed stroke 301 cuts through the entire mesh surface such that visible and occluded faces are selected. Whenever the 2D closed stroke 302 intersects the mesh, the manipulation engine 107 slices the mesh by performing a slicing operation at block 1807, shown in FIG. 18, such as, for example, disclosed by Takeo Igarashi, Satoshi Matsuoka, and Hidehiko Tanaka. 2007. Teddy: a sketching interface for 3D freeform design. In ACM SIGGRAPH 2007 courses (SIGGRAPH '07). ACM, New York, N.Y., USA, Article 21, incorporated herein by reference.

Other slicing operations may be used. For example, as shown in FIGS. 3A to 3D, a fractured anatomical feature is represented by a polygonal mesh comprised of triangular faces. If the face has at least one 3D vertex whose corresponding 2D screen coordinates falls within the 2D screen coordinates of closed stroke 302 it will now be identified by the manipulation engine 107 as belonging to the selected fracture segment 303, as shown in FIG. 18 at block 1807. The selected fracture segment 303, then, may be moved independently from, and relative to, the surrounding anatomical feature 304. Using a two finger panning input gesture to translate and a one finger panning input gesture to rotate the user may manipulate the selected fracture segment 303 to a desired location as depicted in FIG. 3D.

As shown in FIG. 18, at block 1803, the input unit 105 receives the user input gesture and at block 1809 the manipulation engine 107 moves the segment in response to the user input gesture. At block 1801, the motion and final placement of the segment are displayed on the display unit 103.

In further embodiments, a user may repeat segmentation on a fracture element that has already been segmented. For example, a user may segment and manipulate a fracture element, rotate, pan and/or zoom the 3D model, and then segment a portion of the element, as described above. The unselected portion of the element is returned to its original location (i.e., as derived from the CT scans), and the selected portion is segmented from the element. The user may repeat manipulation and segmentation as desired. The user may thereby iteratively segment elements.

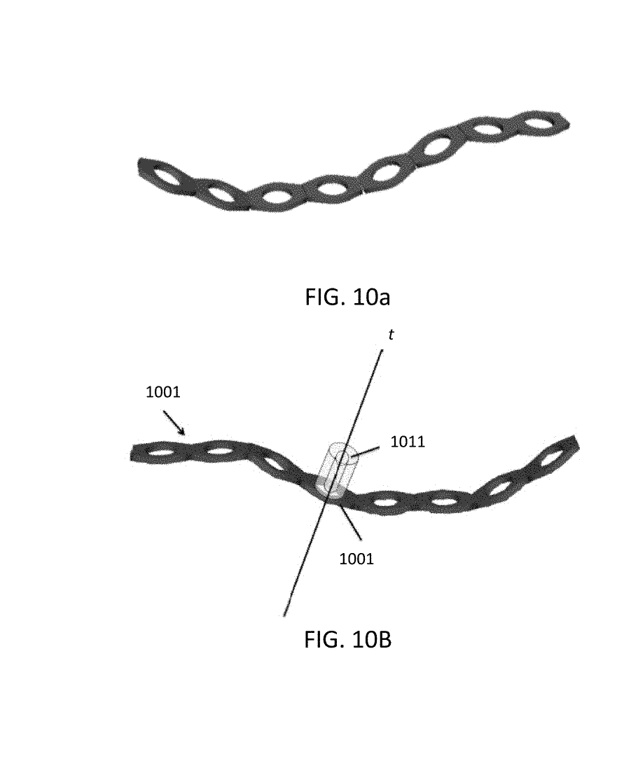

In further aspects, the present systems and methods provide preoperative design of surgical implants, such as, for example, surgical plates and screws. Many surgical treatments call for installation of metal surgical plates in an affected area, such as the surgical plate shown in FIG. 10A. For example, surgical plates are frequently used to stabilise fragmented bone. Surgical plates are preferably stiff to enhance stabilisation. In typical applications, surgical plates require complex bending by hand to effectively treat affected areas. Bending is frequently performed in-vivo. It has been found, however, that such surgical plates are frequently difficult to bend, where bending comprises one or more of: in-plane bending, out-of-plane bending and torquing/twisting. The present systems and methods may assist users to create precisely contoured surgical implants with dimensions corresponding to actual surgical instrument sets.

In aspects, the user may plan placement and configuration of a surgical implant by virtually contouring a 3D model of the surgical implant on the 3D model of the anatomical feature to be treated. After contouring the 3D model, a surgeon may view the model in a 1:1 aspect ratio on the touch screen as a guide to form an actual surgical implant for subsequent use in surgery. Further, in aspects, the digital model of the surgical implant contains sufficient in information for rapid prototyping (also referred to as 3D printing) of a template of the surgical implant or of the actual implant. Where the 3D model is used to generate prototype that will be used as an actual implant, the rapid prototyping method and materials may be selected accordingly. For example, the resulting prototype may be made out of metal.

The printed template may serve as a guide to contour a metal physical implant or further as a drill guide for precise drill and screw placement during surgery. Therefore, pre-surgically planned screw trajectories may be incorporated into the digital surgical implant model to allow rapid prototyping of a pre-contoured template that also contains built-in drill or saw guides for each screw hole in the implant, as herein described in greater detail.

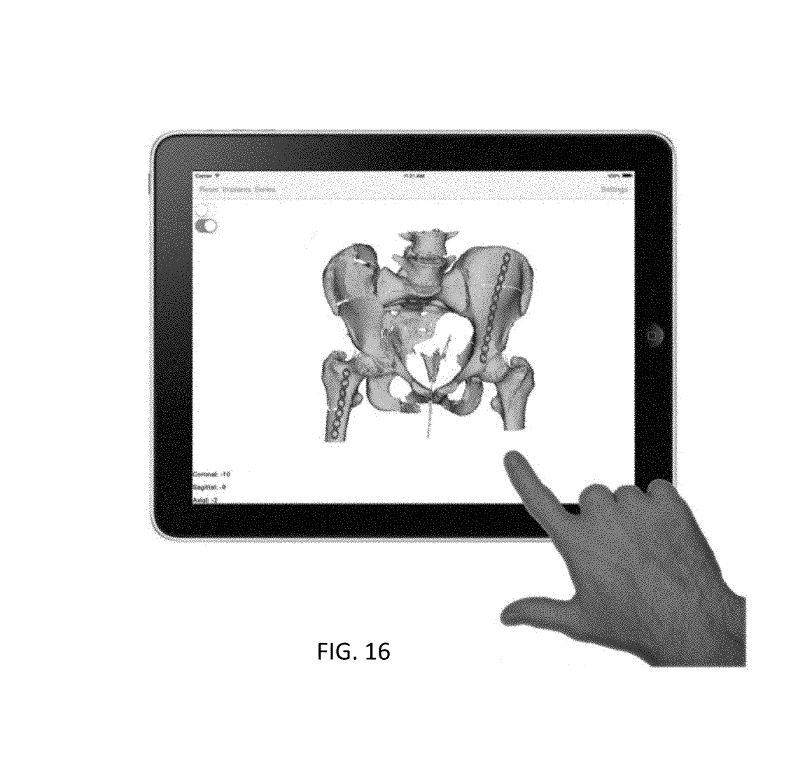

In order to plan placement of surgical implants, the user uses suitable input gestures to manipulate the 3D model of the anatomical features to obtain an appropriate view for planning the surgical implant. The user then indicates that he wishes to plan the surgical implant by, for example, selecting “Implants” in the user interface, as shown in FIG. 16. The user interface may provide further menus and sub-menus allowing the user to select, for example, more specific implant types.

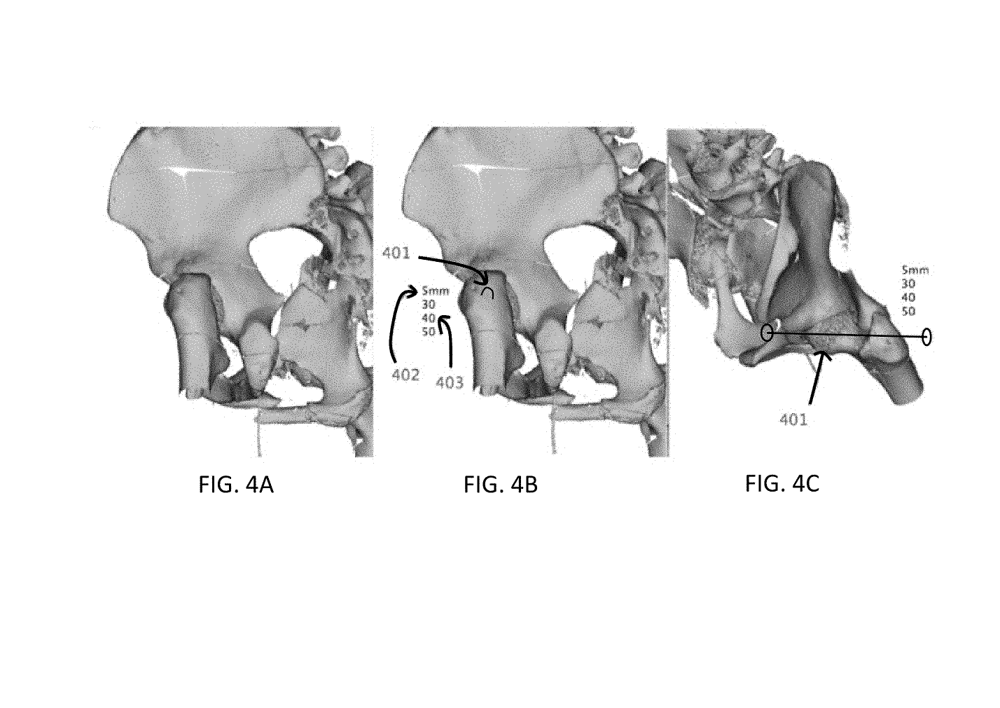

Referring now to FIGS. 4A through 4C, embodiments are shown in which the system provides an intuitive mechanism to plan placement of a drill and screws by determining optimal start points, trajectories, sizes and lengths.

A 3D model of an anatomical feature into which a screw is to be placed is displayed, as previously described. The user may use any of the aforementioned input methods to manipulate the 3D model to find an appropriate view for placing a starting point for screw insertion, as shown in FIG. 4. In embodiments, the user taps touch screen 104 once to establish a start point for a line trajectory 401.

As shown in FIG. 18, the manipulation engine 107 performs operations at block 1811 enabling a user to plan screw and hole placement described above and in greater detail below.

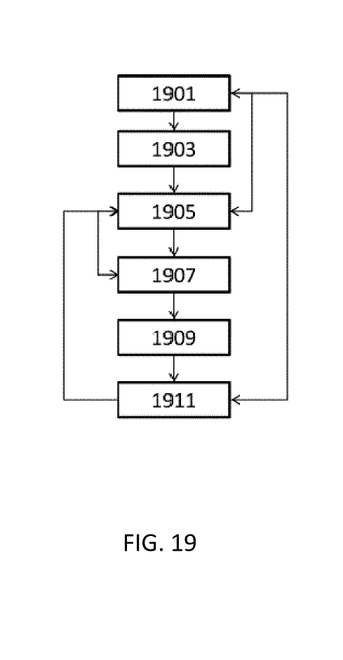

The manipulation engine 107 shown in FIG. 1 converts the 2D touch point on the touch pad 104 to the 3D point on the surface of the 3D model using projection techniques, such as, for example, ray casting and depth buffer lookup, to convert screen coordinates to 3D coordinates to establish a start point for a line trajectory 401 along a view vector perpendicular to the touch screen as illustrated in FIGS. 4B and 4C. The conversion is shown in FIG. 19 at block 1903.

The trajectory 401 having been established, at block 1905 the manipulation engine 107 causes a selection menu to be displayed on the touch screen so that the user may select the length 402 of the screw in the trajectory 401, as well as the angle 403 of the screw relative to either of the orthogonal planes or other screws. At block 1901 the input unit 105 receives the user's selection as a user input gesture and at block 1905 the manipulation engine causes the length to be displayed.

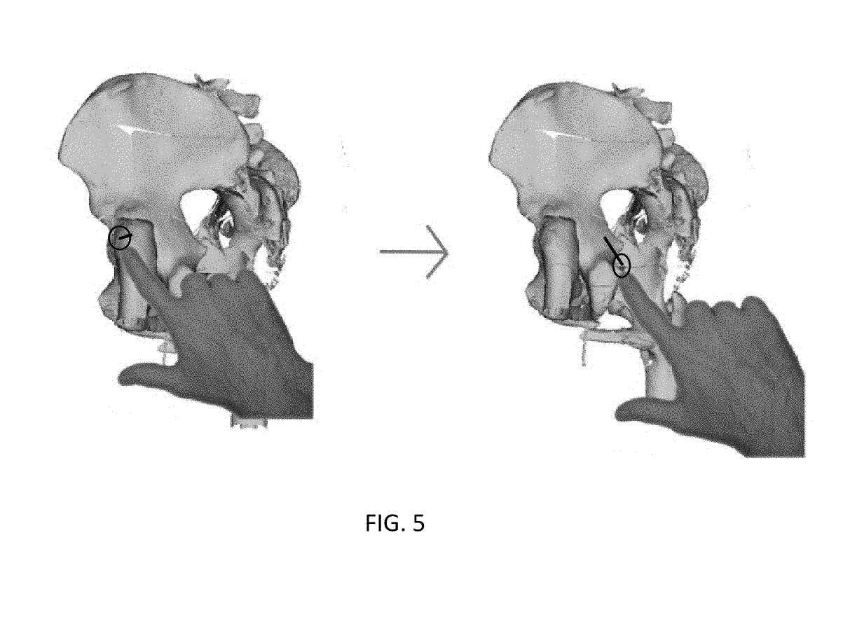

In embodiments, the user may further modify the screw trajectory, as shown in FIG. 5. When the user taps either end point of the line trajectory, at block 1901 the user input 105 relays the gesture to the manipulation engine 107, which liberates the end point, as shown at block 1909. The user can reposition the end point elsewhere on the anatomical feature and redefine the trajectory. At block 1911, the manipulation engine 107 performs the adjustment and at block 1905 causes the adjustment to be displayed. Further, in embodiments, when the user double taps either end point, the screw trajectory is deleted.

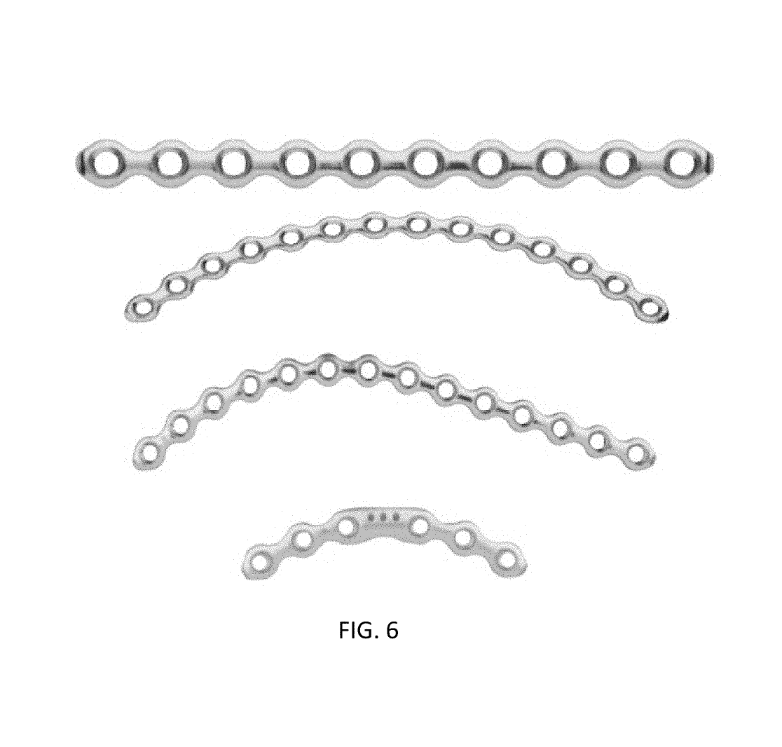

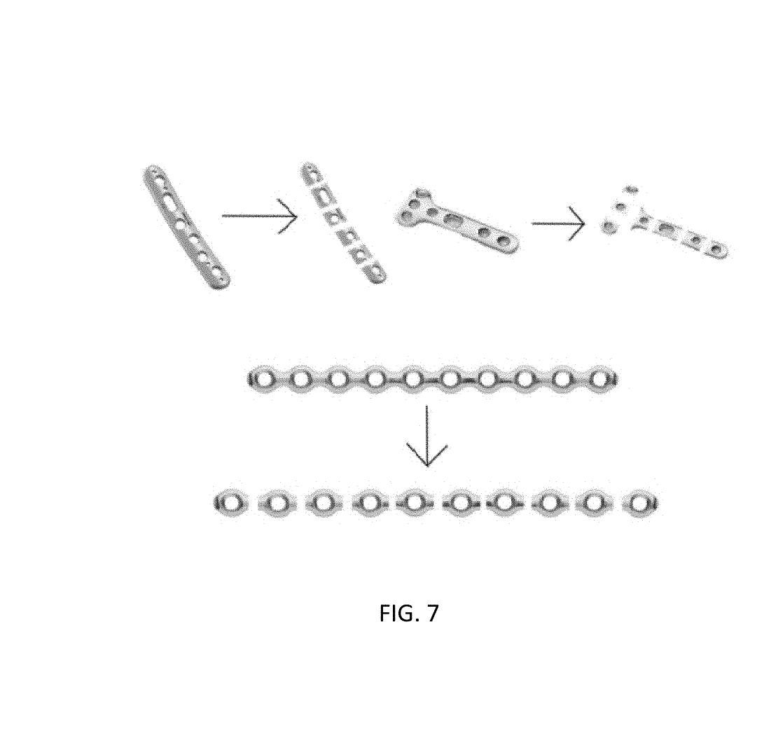

In further embodiments, the user may plan sizing and placement of further surgical implants, such as, for example, surgical plates. In embodiments, 3D models of surgical plates are provided. The 3D models represent surgical plates, such as those shown in FIG. 6. In further embodiments, 3D models, as shown in FIGS. 10A and B, are modelled to represent a string of plate segments, as shown in FIG. 7.

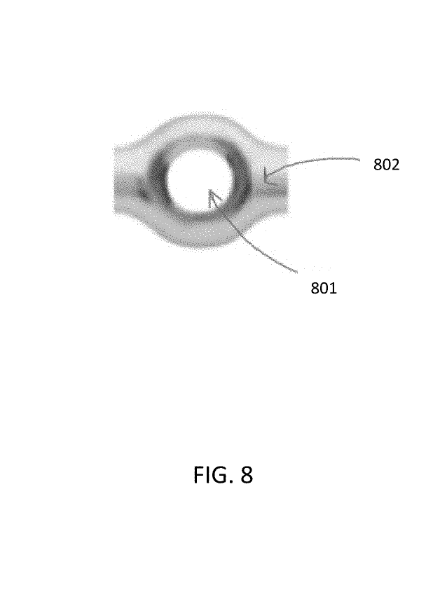

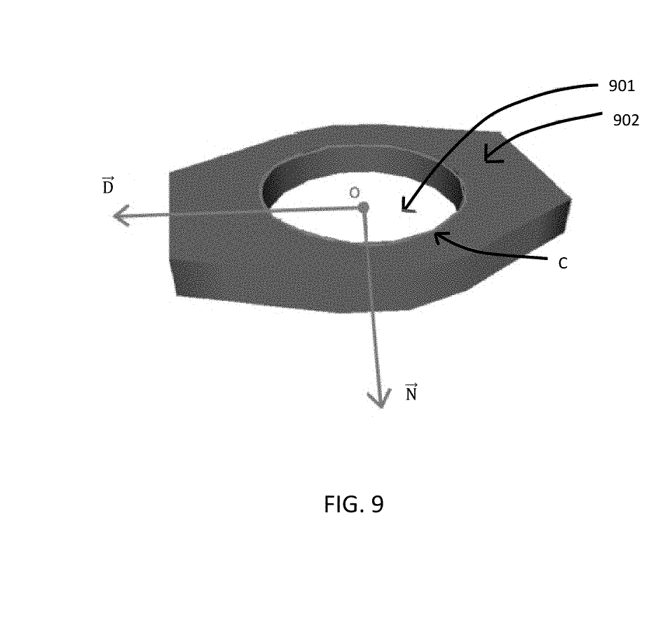

Typically, as shown in FIG. 8, a plate segment comprises a hole 801 and an edge 802 around the hole. It will be appreciated that the size and shape of the hole and the edge may vary between plate designs, as shown in FIGS. 6 and 7. The plate segments are defined in the memory 111 as 3D polygonal models created by available 3D modelling software (not shown), including for example, Autodesk™ Mayan™, Blender™. As illustrated in FIG. 9, a type of plate segment is modelled in 3D. The 3D model of the plate segment has a circular hole 901 and an edge 902 around the hole 901. The plate segment is shown from the bottom. Point O represents the centre of the closed curve C bounding the circular hole 901 at the bottom of the plate segment. Normal vector N is a vector orthogonal to the surface of the plate segment. Vector D is typically perpendicular to normal vector N, and is directed along the longitudinal axis of the plate segment.

It will be appreciated that other types of plates and plate segments may be created, either by the user or by third parties. The user may remodel the size and shape of the hole and shape of the edge for each segment of the plate. Appropriate users may further easily determine a correct position of point O for different hole designs and the direction of a normal vector N and vector D for the different plate segments. Different models may be loaded into the memory 111, for retrieval by the manipulation engine 107.

In still further aspects, the hospital's database 141, as shown in FIG. 1, contains data corresponding to the hospital's actual and/or planned inventories of various surgical implants. The different surgical implant models stored in the memory 111 of the user's database may correspond to actual surgical implants inventoried in the database so that the user can determine whether the surgical implant he is designing is or will be available for the surgery. Other types of inventories, such as, for example, available instruments for performing a surgical operation or the sterilisation status of the available instruments, may be maintained in the database 141 for viewing on the touch screen 104 as a menu option of the user interface. This may enhance the degree to which the user may pre-plan surgical operations.

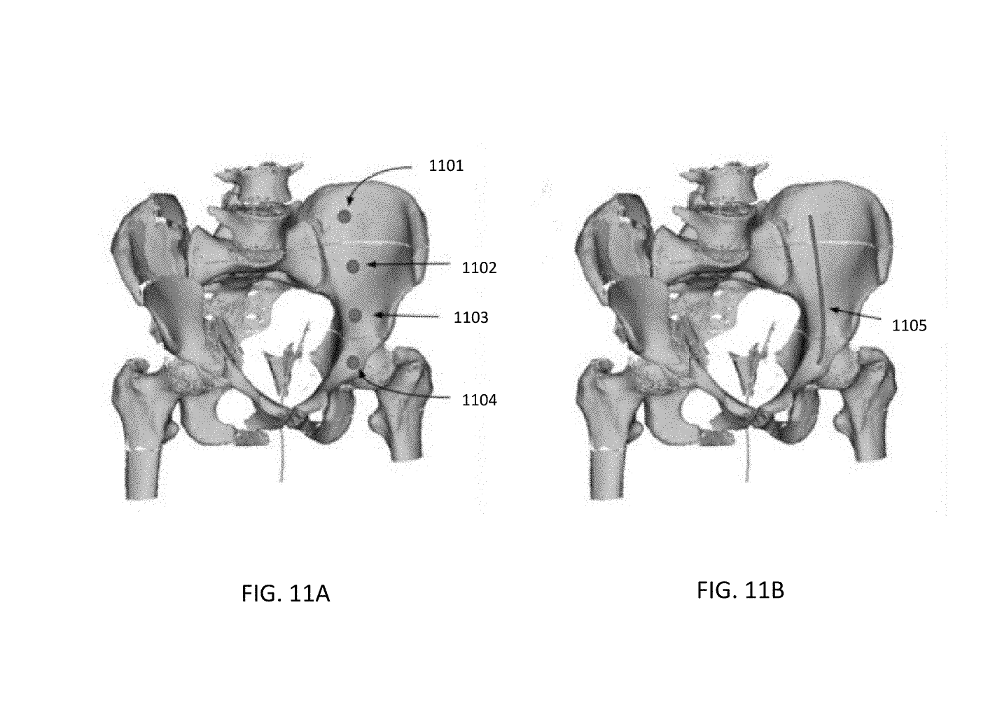

FIGS. 11A to 15 show embodiments of a user interface for planning placement of the previously described plate. The user interface is enabled by various systems and methods described herein. The user interface may further assist users in establishing an optimal selection of plate position, length and contour, as well trajectories and lengths for screws, such as the previously described surgical screws, used to affix the plate to the affected area.

The system provides for automatic and manual virtual manipulation of the model of the surgical plate, including, for example, in-plane, out-of-plane bending and torquing/twisting to contour the plate to the bone surface.

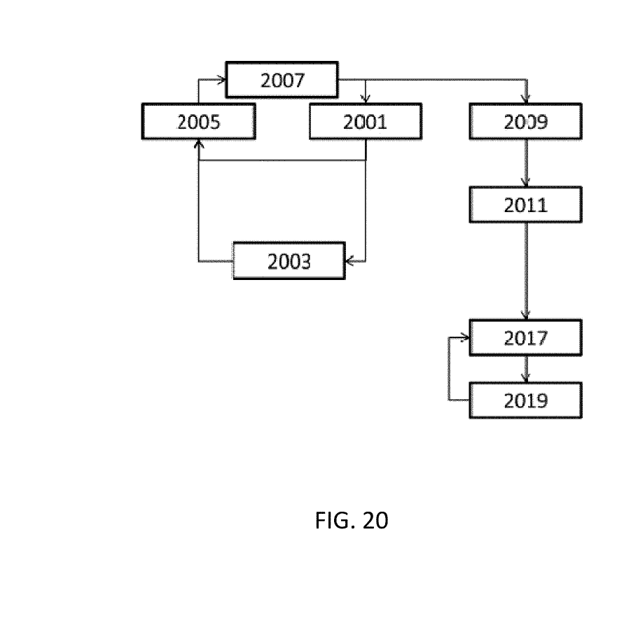

In embodiments, a 3D model is displayed at block 2001, as shown in FIG. 20. At block 2005 the manipulation engine responds to user inputs, as previously described, by rotating, translating and scaling the 3D depiction of the anatomical feature, as previously described to display the desired view at block 2001. The user taps the touch screen 104 once to establish a desired plate start point 1101 as shown in FIG. 11A. The user may then either manually select plate points along a trajectory from the plate start point, or select automatic placement of additional plate points along the trajectory.

In the manual scenario, upon selecting the plate start point 1101, the user again taps the touch screen 104 at other locations to establish next plate points 1102, 1103, 1104 and so on. The number of points may be any number. At block 2007, the manipulation engine 107 converts each of the 2D touch point coordinates to a location on the surface of the 3D model of the anatomical feature, according to previously described techniques. In embodiments, at block 2003 the manipulation engine 107 calculates the shortest geodesic path to define a curve 1105 between points 1101, 1102, 1103 and 1104, as shown in FIGS. 11A and 11B, according to a method, such as, for example, a method invoking a best-fit algorithm, or the method taught by Mitchell et al, “The Discrete Geodesic Problem” (1987) 16:4 Siam J Comput 647, incorporated herein by reference. It will be appreciated that curve 1103 is a discrete curve.

In the automated scenario, upon selecting the plate start point 1101, the user again taps the touch screen 104 at a desired plate end point 1104 to establish an end point for the trajectory. At block 2007, the manipulation engine 107 converts each of the 2D touch point coordinates to a location on the surface of the 3D model of the anatomical feature, as in the manual scenario. In embodiments, at block 2003 the manipulation engine 107 calculates the shortest geodesic path to define a curve 1105 between points 1101 and 1104, as shown in FIGS. 11A and 11B, and as described in the manual scenario.

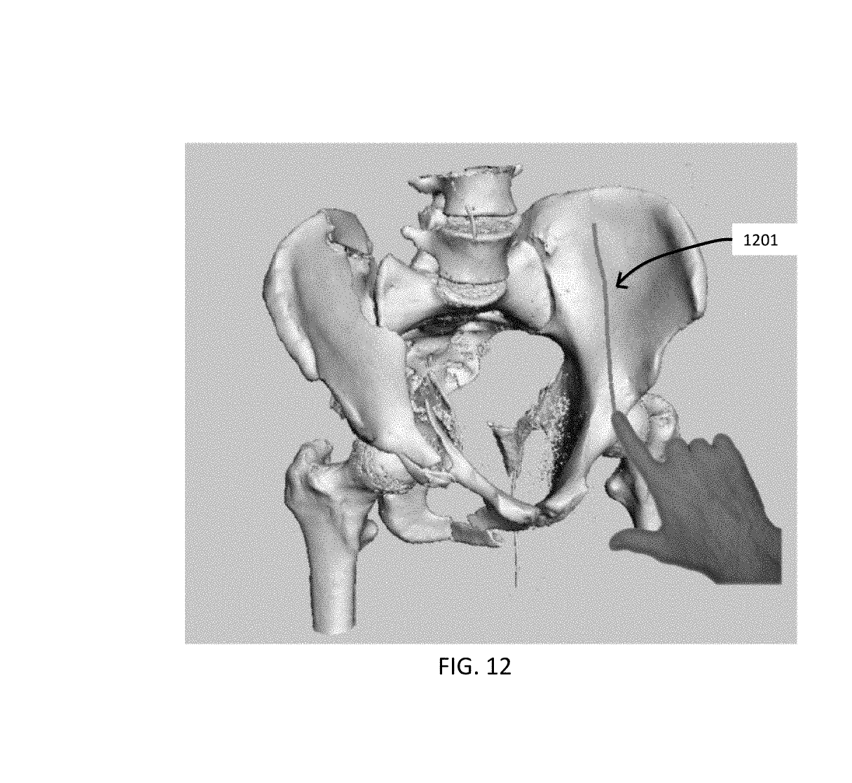

It will be further appreciated that the shortest geodesic path is not always optimal; in embodiments, therefore, the user may alternatively, and preferably selectively, use one-finger panning to draw a customised 2D stroke on the surface of the touch screen 104. At block 2007, the manipulation engine 107 converts each of the 2D stroke coordinates to a location on the surface of the 3D model of the anatomical feature, using known methods as previously described. As a result, a 3D discrete curve 1201 that lies on the surface of the 3D model is created, as shown in FIG. 12.

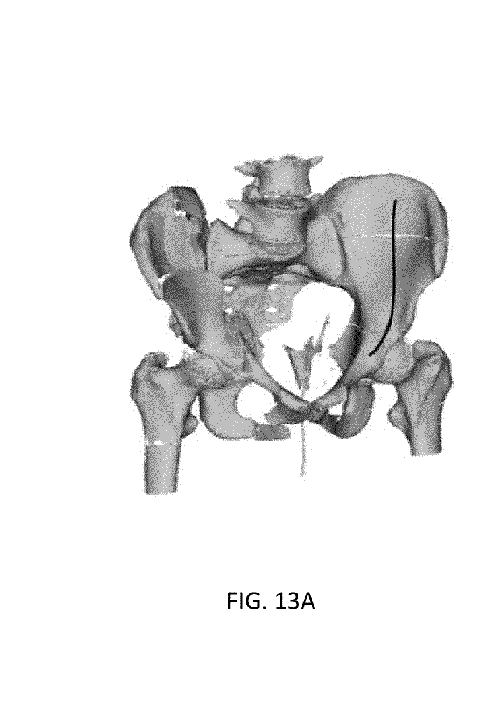

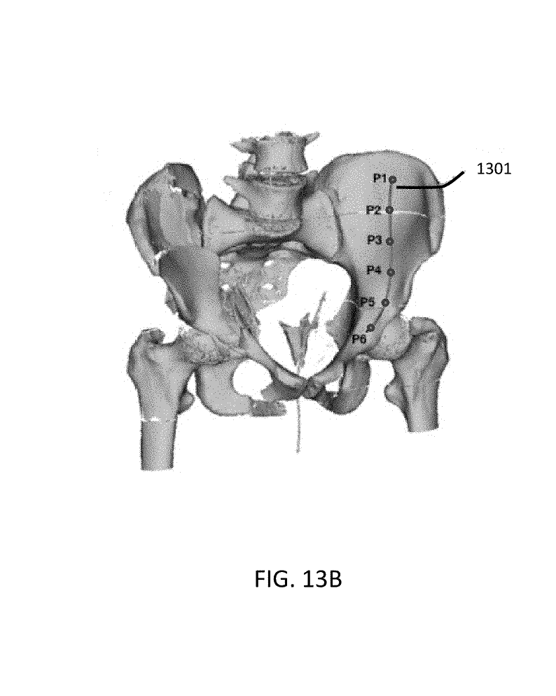

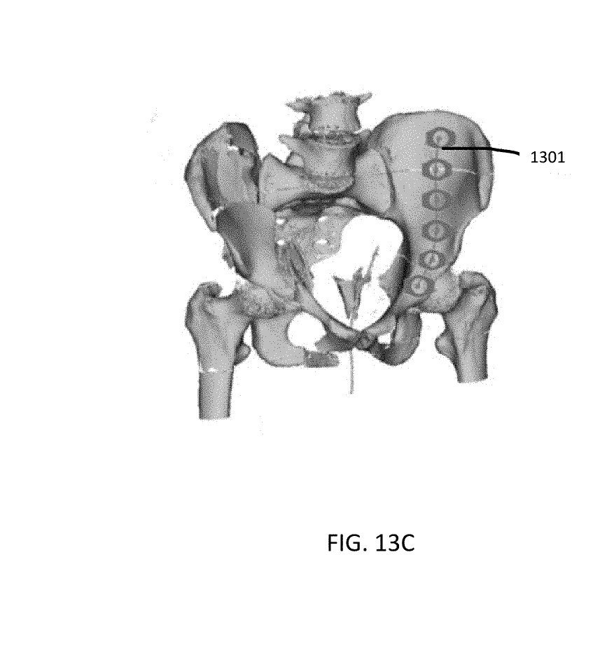

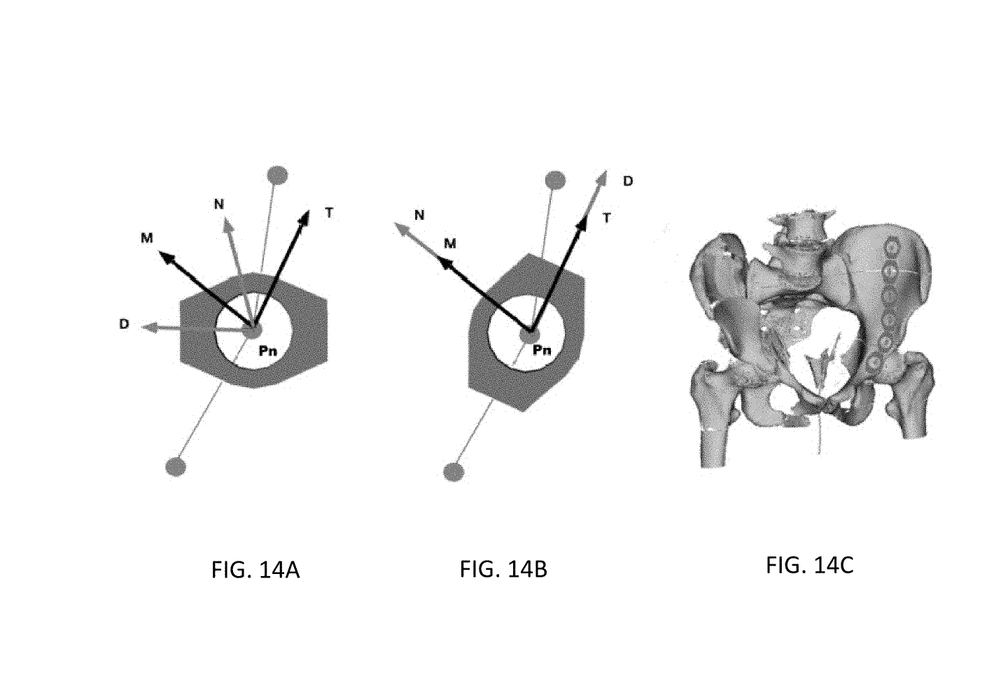

Regardless of the resulting curve, in the automated scenario, the manipulation engine 107 segments the discrete curve 1105 or 1201 into a segmented discrete curve 1301 according to suitable techniques, as shown in FIGS. 13A and 13B. Each point P1, P2 . . . P6 of the segmented discrete curve 1301 may be a location where the hole centres O, shown in FIG. 9, of the plate segments are placed. It will be further appreciated that each point P1 and P6 (or, when the segmented discrete curve 1301 comprises n segments, P1 and Pn+1) may lie at either end point of the segmented discrete curve 1301. Each intermediate points—in this case P2, P3 . . . P5 (or, in embodiments where the segmented discrete curve 1301 comprises n segments, P2 and P(n−1)—could accordingly lie at an intersection between two segments of the segmented discrete curve 1301. During segmenting of the discrete curve at block 2009, the manipulation engine 107 may thus size the line segments to accommodate edges of two selected adjacent plate segments each of whose hole centres is located at either end point of the line segment. As shown in FIG. 13C, the manipulation engine automatically places, at block 2011, and displays, at block 2017, plate segments at every point of the segmented curve 1301. Once the centres of the plate segments are positioned, the manipulation engine automatically contours them by rotating each plate segment to follow the shape of the surface of the anatomical feature along the segmented discrete curve 1301, shown in FIG. 13C. The manipulation engine performs two rotations for each plate segment, as shown in FIGS. 14A and 14B. The first rotation is about the axis defined by the normal vector V; the plate is rotated until plate vector D aligns with vector T, which is the tangent vector to the discrete curve 1103 or 1201 at the point Pn. The second rotation is about the axis defined by the longitudinal axis of the plate; the plate is rotated so that the plate normal vector N aligns with vector M, which is the normal vector of the surface of the anatomical feature at point Pn, as shown in FIG. 14B. After each of the rotations has been performed, a contoured plate as shown in FIG. 14C is provided. In embodiments, the user may delete any plate segment by double tapping it.

Upon manual or automatic placement and alignment of the segments, the manipulation engine may further assign a control point at the hole for each segment. The user may manipulate each control point by any suitable input, in response to which the manipulation engine moves the model of the corresponding segment, for example, in-plane, or along the curve.

In one aspect, the interface may provide an over-sketch function enabling the user to manipulate the surgical plate or segments of the surgical plate, either by moving segments, or by altering the curve along which the segments are located. For example, the user may initiate the over-sketch function by touching the touchscreen over one of the control points and swiping towards a desired location. The manipulation engine reassigns the feature associated to the control point to the new location, and re-invokes any suitable algorithm, as previously described, to re-calculate and adjust the curve and the surgical plate.

The use of the system during surgery has apparent benefits in the context of implant preparation and placement. For example, once a preoperative plan made with the system has been finalised, the manipulation may have generated a 3D model of a surgical implant having a particular set of curvatures, bends and other adjustments. A surgeon, upon conducting the surgery, may refer directly to the system when preparing the actual surgical implant to ensure that the implant is formed as planned. Such a possibility is further enhanced as the surgeon can easily use gesture commands to scale the rendered implant to real-world scale and can rotate the rendered and real-world implants simultaneously to compare them to one another.

The 3D model may enhance or ease fabrication of the physical implant to be used in surgery. Users may view the 3D model of the surgical implant as a guide aiding with conceptualisation for contouring the physical implant, whether preoperatively or in the field. The user may view the model on the touchscreen of her device. In aspects, the interface provides a menu from which the user may select presentation of a preconfigured 1:1 aspect ratio viewing size representing the actual physical dimensions of the surgical implant to be used in surgery. Additional preconfigured views may include the following, for example:

Model—a standard 3D orthographic projection view where user can rotate/scale/translate the model using gestures described previously;

Side—an orthographic projection view from the left and/or right hand side of the plate model;

Front—an orthographic projection view from the front and/or back of the plate model; and

Top—an orthographic projection view from the top and/or bottom of the plate model.

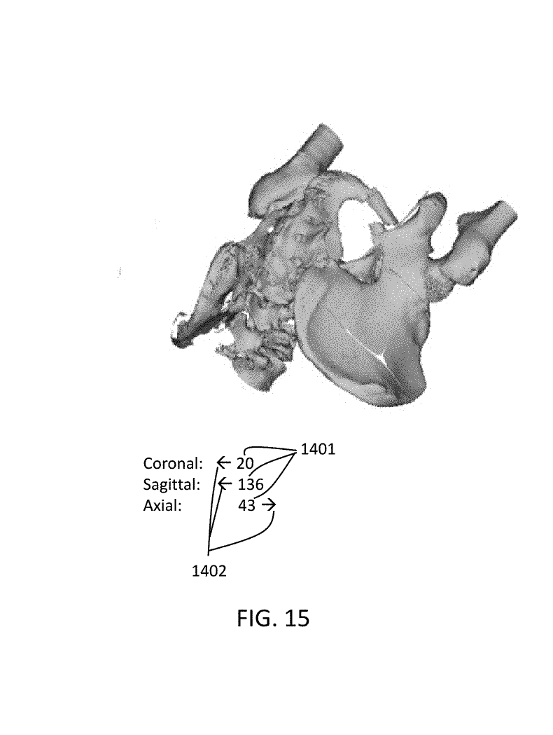

In preferred embodiments, a projection angle icon for the 3D model of the anatomical features is provided and displayed as shown in FIG. 15. The icon displays in real time angles of projection 1401 of the 3D model relative to orthogonal display planes. Arrows 1402 show the direction of rotation of each angle. In preferred embodiments, the angles of projection 1401 displayed are the angles between the orthogonal display planes and the coronal, sagittal and axial planes of the anatomical feature. In still further embodiments, the icon is capable of receiving user input for each of the three provided angles. A user may input into the icon the angles of a desired view. Manipulation engine 107 manipulates the 3D model of the anatomical feature in response to the inputs and causes the display to depict the 3D model at the desired angles. The icon thus enables users to easily record and return to preferred views. For example, a physician may record in advance all views to be displayed during the operating procedure. The views can then be precisely and quickly retrieved during the procedure.

The interface may further enhance pre-operative surgical planning and surgical implant assembly by exporting the 3D models of the surgical implants and anatomical features for use in 3D printing. For example, a “negative” mould of a surgical implant may guide a surgeon in shaping bone grafts during surgery.

The modelling engine may be configured to export digital models in any number of formats suitable for 3D prototyping. The modelling engine may export various types of digital models, such as, for example: anatomic structures, including bone fragments; and surgical implants, including contoured plates, screws and drill guides.

In an exemplary scenario, upon finalisation of a preoperative plan, the modelling engine may export digital models in, for example, a Wavefront .obj file format or STL (StereoLithography) file format. In order to model screws, the manipulating engine obtains the length, trajectory and desired radius for each screw and generates a 3D model (using any of the previously described modelling techniques) of a cylinder with a cap, emulating a screw. The modelling engine exports the 3D model for 3D printing.

Furthermore, the printed plate model can also be utilized as a drill guide for precise drill and screw placement during the surgery. To achieve this, the pre-surgically planned screw trajectories are incorporated into the precisely contoured digital plate model that also contains built-in drill guides for each screw hole in the plate. Overall this may improve surgical accuracy by assisting the user to avoid important anatomical structures, improve efficiency by reducing surgical steps, reduce the number of standard instruments needed, reducing instruments to re-sterilize, reducing wastage of implants, and facilitates faster operating room turnover.

Referring now to FIG. 10B, an exemplary model of drill guide incorporated in the digital model of a surgical plate 1001 is shown. In embodiments, the manipulation engine models drill guides for the surgical plate about each location requiring a screw. The manipulation engine models each drill guide as a cylindrical sleeve 1011 abutting the segment 1005 of the surgical plate 1001 opposite any anatomical feature (not shown) to which the plate is to be applied or attached. The cylindrical sleeve 1011 is coaxially aligned with the preplanned corresponding screw trajectory, shown by the line t, and which is described above in greater detail. The manipulation engine obtains a user input for each or all of the drill guides indicating a desired drill diameter and cylindrical sleeve length, and accordingly generates the drill guide model. The modelling engine exports the modelled drill guide for 3D printing, as previously described.

3D printed drill guides printed from 3D models generated according to the systems and methods herein, such as discussed with reference to FIG. 10B, preferably demonstrate sufficient biomechanical strength to receive appropriately sized drill bits for the particular application required. Printed drill guides, which may be principally constructed of various plastics, may further be lined with metal sleeves to reduce wear by reinforcing the sleeves. Drill guides may either be unitised with the printed surgical plate template, or be screwed in to the printed surgical plate in modular fashion. Modular drill guides allow the printed surgical plate template to be inserted separately into difficult to reach anatomical areas thereby causing minimal trauma to important surrounding soft tissue structures. The drill guides can then be screwed into the surgical plate model with the correct trajectory after the surgical plate template is positioned anatomically.

It will be appreciated that the system may be provided on a mobile tablet device. By its nature, such a device is easily transportable and may be used in a surgical setting to augment the surgeon's tools available therein. For example, a surgeon could utilize the system before, during or both before and during surgery. An illustrative example enables a surgeon to have a more thorough view of a particular bone fracture using the system than the surgeon could otherwise have by simply looking directly at a bone fracture within a patient's body.

It will be further appreciated that the preoperative screw and plate positions determined using the aforementioned methods can be stored in the memory 111 for post-operative analysis. In embodiments, a post-operative 3D model is generated by the modelling engine from post-operative CT datasets as heretofore described. The user may recall the preoperative screw and plate positions from the memory 111, so that the positions are superimposed over the post-operative 3D model. It will be appreciated that the accuracy of the surgical procedure can thus be gauged with respect to the planned procedure.

Although the illustrated embodiments have been described with particular respect to preoperative planning for orthopaedic surgery, it will be appreciated that a system and method for interactive 3D surgical planning may have many possible applications outside of orthopaedic trauma. Exemplary applications include, but are not limited to, joint replacement surgery, deformity correction and spine surgery, head and neck surgery, oral surgery and neurosurgery.

It will be further appreciated that the embodiments described may provide educational benefits, for example as a simulation tool to train resident and novice surgeons. Further, the embodiments may enable improved communication between surgeons and patients by offering enhanced visualisation of surgical procedures.

Orthopaedic implant manufacturing and service companies will appreciate that the foregoing embodiments may also provide a valuable marketing tool to display implants and technique guides, or to employees.

It will further be appreciated that the embodiments described may be used to train X-ray technologists to optimise patient positioning and X-ray projection selection.

It will further be appreciated that the above-described embodiments provide techniques to provide rapid access to automated segmentation allowing active participation in planning, design and implantation of patient-specific implants, including “lasso” segmentation, facilitating screw hole planning, drill-guide modeling, and contouring a modeled implant plate. Further, the embodiments may be applicable to a range of anatomical features, including, but not limited to hips and knees.

It will further be appreciated that that the above-described embodiments provide a unified simulation system, optimized for use on mobile touch-screen devices, allowing users, such as surgeons and medical device engineers to work in parallel during the design of patient-matched implants and to contribute to reducing the overall temporal and financial cost of the manufacture thereof. Embodiments described above thus provide a unified platform for 3D surgical planning and implant design which may enhance communication between surgeons and engineers.

Although the invention has been described with reference to certain specific embodiments, various modifications thereof will be apparent to those skilled in the art without departing from the spirit and scope of the invention as outlined in the claims appended hereto. The entire disclosures of all references recited above are incorporated herein by reference.

What is claimed is:

1. A system for generating a two-dimensional rendering of a three-dimensional model of an anatomical feature in response to a user input action from a user, the system comprising:
a display unit for displaying a plurality of parameters, the plurality of parameters corresponding to Hounsfield values;
an input unit for receiving the user input action from the user, the input action comprising selecting at least one of the plurality of parameters, each corresponding to one of the Hounsfield values; and
a manipulation engine, executed on a computing device, configured to:
retrieve a subset of imaging data corresponding to the at least one of the plurality of parameters and generate a three-dimensional model of the anatomical feature therefrom; and
generate a two-dimensional rendering of the three-dimensional model for display on the display unit.

2. The system of claim 1, wherein the plurality of parameters correspond to categories of anatomical features.

3. The system of claim 1, wherein the subset of imaging data comprises pixels having greyscale values.

4. The system of claim 3, wherein the subset of imaging data further comprises two-dimensional coordinates of the pixels.

5. The system of claim 4, wherein the manipulation engine is further configured to calculate three-dimensional coordinates associated with each of the pixels.

6. The system of claim 1, wherein the manipulation engine is further configured to apply one or more volume rendering techniques to generate the three-dimensional model.

7. The system of claim 1, wherein the three-dimensional model is a point cloud model.

8. The system of claim 1, wherein the three-dimensional model is a polygon mesh model.

9. The system of claim 1, wherein the manipulation engine is further configured to export the three-dimensional model to a three-dimensional printing device for printing.

10. A method for generating a two-dimensional rendering of a three-dimensional model of an anatomical feature in response to a user input action from a user, the method comprising:
displaying a plurality of parameters, the parameters corresponding to Hounsfield values;
receiving the user input action from the user, the input action comprising selecting at least one of the plurality of parameters each corresponding to one of the Hounsfield values associated with the anatomical feature;
retrieving a subset of imaging data corresponding to the at least one of the plurality of parameters;
generating a three-dimensional model of the anatomical feature using the subset of imaging data; and
generating a two-dimensional rendering of the three-dimensional model.

11. The method of claim 10, wherein the plurality of parameters correspond to categories of anatomical features.

12. The method of claim 10, wherein the subset of imaging data comprises pixels having greyscale values.

13. The method of claim 12, wherein the subset of imaging data further comprises two-dimensional coordinates of the pixels.

14. The method of claim 13, wherein generating the three-dimensional model comprises calculating three-dimensional coordinates associated with each of the pixels.

15. The method of claim 10, wherein generating the three-dimensional model comprises applying one or more volume rendering techniques.

16. The method of claim 10, wherein the three-dimensional model is a point cloud model.

17. The method of claim 10, wherein the three-dimensional model is a polygon mesh model.

18. The method of claim 10, further comprising exporting the three-dimensional model to a three-dimensional printing device for printing.
